(19) United States
(12) Patent Application Publication
(10) Pub. No.: US 2025/0381345 A1
(43) Pub. Date: Dec. 18, 2025

(54) TIMELY ALERT OF IMPENDING EMPTYING OF MEDICAMENT STORAGE

(71) Applicant: INSULET CORPORATION, Acton, MA (US)

(72) Inventors: Gregory STERN, Lexington, MA (US); Joon Bok LEE, Acton, MA (US); William WHITELEY, Knoxville, TN (US)

(21) Appl. No.: 19/302,842

(22) Filed: Aug. 18, 2025

Related U.S. Application Data
(63) Continuation-in-part of application No. 16/780,278, filed on Feb. 3, 2020.

(51) Int. Cl. A61M 5/172; G16H 20/17

(52) U.S. Cl. CPC A61M 5/1723; G16H 20/17; A61M 2230/201

(57) ABSTRACT

A medicament delivery system may provide timely notice of when a medicament storage for the medicament delivery system is to be empty or near empty or medicament. The system may calculate whether the medicament in the medicament storage will run out during a future interval. The future interval may be an interval of hours in the future. This enables the user to receive early notice of the impending emptying of the medicament storage so as to be to respond the impending emptying in a timely manner. For instance, an alert, such as an alarm, may be generated and sent to the user, and the user may respond, such as be refilling the medicament storage or replacing the medicament storage.

### CROSS-REFERENCE TO RELATED APPLICATIONS

This application is a continuation in part of U.S. application Ser. No. 16/780,278, filed on Feb. 3, 2020.

### BACKGROUND

Insulin held in insulin reservoirs of automated drug delivery devices is often wasted. This is due in part to excessive amounts of insulin being present in the insulin reservoirs. As a result, not all of the insulin in reservoir may be used before the insulin must be discarded. Typically, the insulin in such reservoirs is only suitable for use for a period of three days. There is a risk of infection at the injection site if the insulin is not discarded in a timely fashion. Given that insulin is expensive, this discarding of insulin is especially problematic.

Insulin reservoirs may be filled to standardized levels for all users. Unfortunately, this approach may result in insulin reservoirs containing excessive insulin that needs to be discarded for many users. Insulin reservoirs may be filled to levels that err on being excessive so as to not run out of insulin for a specified period. This problem can be especially acute for users that require less insulin than normal. In other instances, the amount of insulin filled in the insulin reservoir may be too little. This requires the reservoir to be filled frequently, which can result in unnecessary loss of insulin due to priming as part of the refilling.

Another challenge is that a user of an automated drug delivery device may not be aware of when a drug storage for the device is going to be empty due to delivery of all or substantially all of the drug in the drug storage or may only be informed when little drug is left. As a result, the user may not respond to the impending emptying of the drug storage or may not have sufficient time to respond to the impending emptying of the drug storage.

### SUMMARY

In accordance with an inventive facet, a medicament delivery system for delivering a medicament to a user may include a medicament storage for storing the medicament and an output element for generating output to the user. The medicament delivery system also may include a non-transitory storage medium storing programming instructions and one or more processors for executing the programming instructions. Executing the programming instructions may cause the one or more processors to determine an amount of the medicament that is stored in the medicament storage and to determine a quantity of the medicament that will delivered to the user over a period by the medicament delivery system based at least in part on a current analyte level of the user. Executing the programming instructions may further cause the one or more processors to determine whether the quantity of medicament that will be delivered to the user over the period by the medicament delivery system exceeds the amount of medicament that is stored in the medicament storage and to generate output on the output element where it is determined that the quantity of medicament that will be delivered to the user over the period by the medicament delivery system exceeds the amount of medicament that is stored in the medicament storage.

The determining of the quantity of the medicament that will delivered to the user over a period by the medicament delivery system may be based in part on a difference between the current analyte level of the user and a target analyte level of the user. The determining of the quantity of the medicament that will delivered to the user over a period by the medicament delivery system may be additionally based on a quantity of medicament that has been delivered to the user that still has an effect on the analyte level of the user. The analyte level may be a glucose level of the user. The medicament may be insulin, and the quantity of medicament that has been delivered to the user that still has an effect on the analyte level of the user may be the insulin on board (IOB). The determining of the quantity of the medicament that will delivered to the user over a period by the medicament delivery system may be based in part on a total medicament delivered to the user over a prior period. The determining of the quantity of the medicament that will delivered to the user over a period by the medicament delivery system may be based in part on a ratio of basal insulin delivered to the user over a prior period to a total of insulin delivered to the user over the prior period.

In accordance with another inventive facet, an insulin delivery system for delivering insulin to a user may include a storage for storing the insulin and an output element for generating output to the user. The insulin delivery system may further include a non-transitory storage medium storing programming instructions and one or more processors for executing the programming instructions. The executing of the programming instructions by the one or more processors may cause the one or more processors to receive from a sensor a signal indicative of an amount of insulin stored in the storage and to determine from the signal how much insulin is stored in the storage. The executing of the programming instructions by the one or more processors may further cause the one or more processors to determine a quantity of the insulin that will delivered to the user over a period by the insulin delivery system based at least in part on a current glucose level of the user, a target glucose level of the user and insulin on board for the user and to generate output on the output element where it is determined that the quantity of insulin that will be delivered to the user over the period by the insulin delivery system exceeds the amount of insulin that is stored in the insulin storage.

The one or more processors in determining the quantity of insulin that will be delivered over the period may determine an amount of insulin that is needed to bring the current glucose level of the user to a target level of the user. The one or more processors in determining the quantity of insulin that will be delivered over the period may reduce the determined amount of insulin that is needed to bring the current glucose level of the user to a target level of the user by the insulin on board (IOB) for the user. The one or more processors in determining the quantity of insulin that will be delivered over the period may add an anticipated amount of basal insulin to be delivered over the period based on a total daily insulin (TDI) value derived from recent insulin delivery data for the user. The anticipated amount of basal insulin to be delivered over the period based on the TDI value for the user may be set as half of the TDI value for the user. The anticipated amount of basal insulin to be delivered over the period based on the TDI value for the user may be set as a product of multiplying the TDI value for the user by a proportionality value that is indicative of what portion of the TDI for the user are basal insulin deliveries in the recent insulin delivery data for the user. The output may be a notification that the insulin in the storage will be fully consumed by a specified date.

In accordance with another inventive facet, a medicament delivery system for delivering a medicament to a user may include a medicament storage for storing the medicament, an output element for generating output to the user, and a non-transitory storage medium storing programming instructions. The medicament delivery system also may include one or more processors for executing the programming instructions to cause the one or more processors to determine a quantity of medicament that remains in the medicament storage and to determine a quantity of the medicament will be delivered from the medicament storage over an interval of at least an hour in duration. The determining of the quantity of the medicament that will be delivered from the medicament storage over the interval may include determining a quantity of basal medicament that will be delivered over the interval as basal medicament deliveries; determining a quantity of medicament for a correction bolus that would bring an analyte level of the user to a target level; summing the determined quantity of basal medicament that will be delivered over the interval as basal medicament deliveries with the detrained quantity for the correction bolus to determine a sum used in determining the quantity of the medicament will be delivered from the medicament storage over the interval; comparing the quantity of the medicament that will be delivered from the medicament storage over the interval to the quantity of the medicament that remains in the medicament storage, where the quantity of the medicament that will be delivered from the medicament storage over the interval is greater than the quantity of the medicament that remains in the medicament storage, generating a notification to the user; and where the quantity of the medicament that will be delivered from the medicament storage over the interval is not greater than the quantity of the medicament that remains in the medicament storage, taking no additional action.

The medicament may be insulin and the analyte level may be a glucose level. The determining of the quantity of basal medicament that will be delivered over the interval as basal medicament deliveries may include analyzing recent basal medicament delivery history of the user to determine the quantity of basal medicament that will be delivered over the interval as basal medicament deliveries. The determining of the quantity of basal medicament that will be delivered over the interval as basal medicament deliveries may include calculating an average total daily medicament for the user from the recent basal medicament delivery history of the user and determining a daily quantity of the basal medicament that will be delivered over the interval as fraction of the average total daily medicament for the user. The medicament may be insulin, and the fraction may be one of either one half or a fraction derived from the recent basal medicament delivery history. The determining of the quantity of the medicament that will delivered from the medicament storage over the interval may further include subtracting from the sum quantities of medicament that have been delivered to the user that may still affect the analyte level of the user.

### BRIEF DESCRIPTION OF THE DRAWINGS

FIG. 1 depicts a block diagram of a drug delivery system suitable for practicing exemplary embodiments.

FIG. 2 depicts a block diagram showing options for replenishing an expired insulin reservoir.

FIG. 3 depicts a diagram of types of devices suitable for practicing methods described herein.

FIG. 4 depicts a block diagram of a device suitable for practicing methods described herein.

FIG. 5 depicts a flowchart showing illustrative steps for determining actual insulin daily deliveries for a user.

FIG. 6 depicts a flowchart showing illustrative steps for estimating an amount of insulin needed to fill the insulin reservoir for a user.

FIG. 7 depicts a flowchart showing illustrative steps for determining an estimate of daily bolus needs for a user.

FIG. 8 depicts a flowchart showing illustrative steps for determining an amount of time remaining before insulin in the insulin reservoir will be consumed based on average insulin needs of the user.

FIG. 9 depicts a flowchart of illustrative steps that may be performed in exemplary embodiments to provide an early notification that the medicament delivery will run out of medicament within an interval.

FIG. 10 depicts a flowchart of illustrative steps that may be performed in exemplary embodiments to determine a quantity of medicament in a medicament storage.

FIG. 11 depicts a flowchart of illustrative steps that may be performed in exemplary embodiments to determine an anticipated quantity of medicament delivery over an interval per a first option.

FIG. 12 depicts a flowchart of illustrative steps that may be performed in exemplary embodiments to determine an anticipated quantity of medicament delivery over the interval per a second option.

FIG. 13 depicts a flowchart of illustrative steps that may be performed in exemplary embodiments to determine an anticipated quantity of medicament delivery over the interval per a third option.

FIG. 14 depicts a flowchart of illustrative steps that may be performed in exemplary embodiments to determine anticipated basal medicament requirements over the interval per a first option.

FIG. 15 depicts a flowchart of illustrative steps that may be performed in exemplary embodiments to determine the anticipated basal medicament requirements over the interval per a second option.

FIG. 16 depicts a flowchart of illustrative steps that may be performed in exemplary embodiments to determine the anticipated additional medicament requirements to address the analyte level of the user being above a target level.

### DETAILED DESCRIPTION

Exemplary embodiments may provide an improved approach to automated insulin delivery by more accurately estimating the total daily insulin (TDI) of a user. As a result, less insulin is wasted by the delivery system, and the estimate of TDI more closely matches a user's actual daily insulin needs. Hence, the user need not refill the insulin reservoir excessively or need not fret unnecessarily about running out of insulin prematurely. The estimate relies on the history of actual automated insulin deliveries and thus reflects the actual insulin delivered to the user more accurately than conventional approaches.

Exemplary embodiments may use this more accurate estimate of TDI to determine how much insulin needs to be filled into an insulin reservoir. The TDI may be used to estimate daily basal insulin needs of a user. The estimate of daily basal insulin for the user may be added to an estimate of daily bolus insulin needs to obtain an estimate of total daily insulin needs for the user. The estimate of daily bolus needs is obtained from the actual bolus insulin delivery history of the user. Once the estimate of daily insulin needs for the user is determined, a determination of how many days of insulin are to be held in the reservoir is made. The total amount of insulin to be contained in the reservoir is then calculated by multiplying the number of days of insulin to be held in the reservoir by the estimate of daily insulin needs of the user. The reservoir may be filled with the calculated amount of insulin. This approach provides a more accurate estimate of the amount of insulin needed in the reservoir for the designated number of days than conventional approaches and avoids both needing to refill the reservoir prematurely or overfilling the reservoir so that costly insulin is wasted.

Exemplary embodiments also may provide an accurate estimate of the amount of time before a reservoir needs to be replenished. In these exemplary embodiments, the user may be asked to state the quantity of insulin that filled the reservoir at the time of filling the reservoir. Based on this quantity, the exemplary embodiments may estimate the time remaining before the insulin expires using the user's actual insulin delivery history rather than an estimate based solely on the basal insulin delivery amount or other clinical parameters that may be inaccurate. The time remaining may be determined based on the estimate of user's daily insulin needs discussed above.

Exemplary embodiments may provide timely notice of when a medicament storage for a medicament delivery system is to be empty or substantially empty of medicament. The exemplary embodiments may calculate whether the medicament in the medicament storage will run out during a future interval. The future interval may be an interval of hours in the future. This enables the user to receive early notice of the impending emptying of the medicament storage so as to be to respond the impending emptying in a timely manner. For instance, an alert, such as an alarm, may be generated and sent to the user, and the user may respond, such as be refilling the medicament storage or replacing the medicament storage.

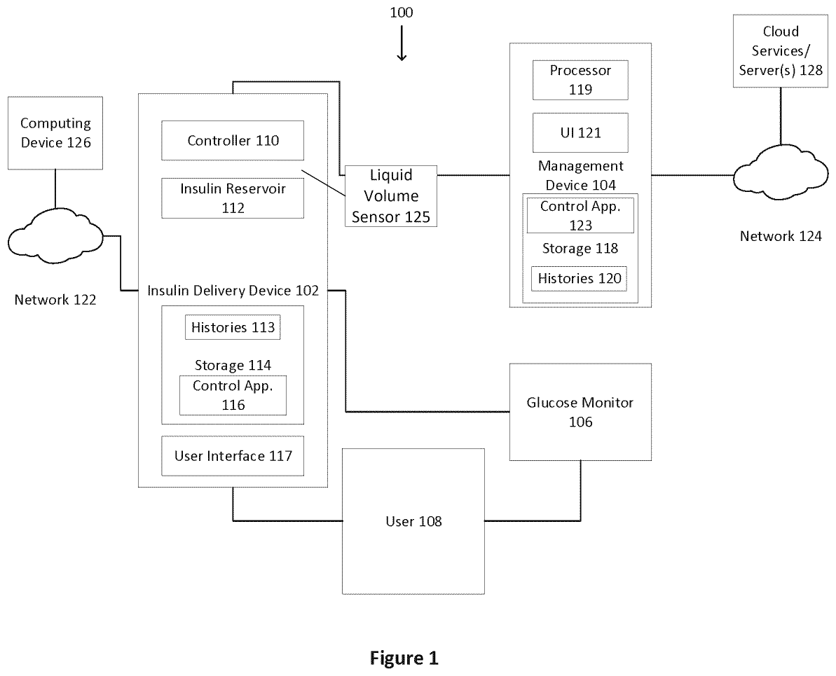

FIG. 1 depicts an illustrative drug delivery system 100 (also referred to more generally as a medicament delivery system) that is suitable for delivering insulin to the user 108 in an exemplary embodiment. The drug delivery system 100 includes a medicament delivery device, such as an insulin delivery device 102. The insulin delivery device 102 may be a wearable device that is worn on the body of the user 108. The insulin delivery device 102 may be directly coupled to a user (e.g., directly attached to a body part and/or skin of the user 108 via an adhesive or the like). In an example, a surface of the insulin delivery device 102 may include an adhesive to facilitate attachment to the user 108.

The insulin delivery device 102 may include a controller 110. The controller 110 may be implemented in hardware, software, or any combination thereof. The controller 110 may, for example, be a microprocessor, a logic circuit, a field programmable gate array (FPGA), an application specific integrated circuit (ASIC) or a microcontroller coupled to a memory. The controller 110 may maintain a date and time as well as other functions (e.g., calculations or the like). The controller 110 may be operable to execute a control application 116 stored in the storage 114 that enables the controller 110 to direct operation of the insulin delivery device 102. The storage 114 may hold histories 113 for a user, such as a history of automated insulin deliveries, a history of bolus insulin deliveries, meal event history, exercise event history and the like. In addition, the controller 110 may be operable to receive data or information. The storage 114 may include both primary memory and secondary memory. The storage may include random access memory (RAM), read only memory (ROM), optical storage, magnetic storage, removable storage media, solid state storage or the like.

The insulin delivery device 102 may include a medicament storage, such as an insulin reservoir 112, for storing a medicament, such as insulin, for delivery to the user 108 as warranted. A liquid volume sensor 125 may be provided to sense the volume of the liquid medicament in the medicament storage, e.g., the insulin reservoir 112. The liquid volume sensor 125 may be positioned in the medicament storage. A fluid path to the user 108 may be provided, and the insulin delivery device 102 may expel the insulin from the insulin reservoir 112 to deliver the insulin to the user 108 via the fluid path. The fluid path may, for example, include tubing coupling the drug delivery device 102 to the user 108 (e.g., tubing coupling a cannula to the insulin reservoir 112).

There may be one or more communications links with one or more devices physically separated from the insulin delivery device 102 including, for example, a management device 104 of the user and/or a caregiver of the user and/or a glucose monitor 106. The communication links may include any wired or wireless communication link operating according to any known communications protocol or standard, such as Bluetooth®, Wi-Fi, a near-field communication standard, a cellular standard, or any other wireless protocol The insulin delivery device 102 may also include a user interface 117, such as an integrated display device for displaying information to the user 108 and in some embodiments, receiving information from the user 108. The user interface 117 may include a touchscreen and/or one or more input devices, such as buttons, knob or a keyboard.

The insulin delivery device 102 may interface with a network 122. The network 122 may include a local area network (LAN), a wide area network (WAN) or a combination therein. A computing device 126 may be interfaced with the network, and the computing device may communicate with the insulin delivery device 102. The role that the computing device 126 may play in the exemplary embodiments will be described in more detail below.

The drug delivery system 100 may include a glucose monitor 106 for sensing the blood glucose concentration levels of the user 108. The glucose monitor 106 may provide periodic blood glucose concentration measurements and may be a continuous glucose monitor (CGM), or another type of device or sensor that provides blood glucose measurements. The glucose monitor 106 may be physically separate from the insulin delivery device 102 or may be an integrated component thereof. The glucose monitor 106 may provide the controller 110 with data indicative of measured or detected blood glucose levels of the user 108. The glucose monitor 106 may be coupled to the user 108 by, for example, adhesive or the like and may provide information or data on one or more medical conditions and/or physical attributes of the user 108. The information or data provided by the glucose monitor 106 may be used to adjust drug delivery operations of the insulin delivery device 102.

The drug delivery system 100 may also include the management device 104. The management device 104 may be a special purpose device, such as a dedicated personal diabetes manager (PDM) device. The management device 104 may be a programmed general purpose device, such as any portable electronic device including, for example, a dedicated controller, such as processor, a smartphone, or a tablet. The management device 104 may be used to program or adjust operation of the drug delivery device 102 and/or the sensor 104. The management device 104 may be any portable electronic device including, for example, a dedicated controller, a smartphone, or a tablet. In the depicted example, the management device 104 may include a processor 119 and a storage 118. The processor 119 may execute processes to manage a user's blood glucose levels and for control the delivery of the drug or therapeutic agent to the user 108. The processor 119 may also be operable to execute programming code stored in the storage 118. For example, the storage may be operable to store one or more control applications 123 for execution by the processor 119. The storage 118 may store the control application 123, histories 120 like those described above for the insulin delivery device 102 and other data and/or programs.

The management device 104 may include a user interface 121 for communicating with the user 108. The user interface may include a display, such as a touchscreen, for displaying information. The touchscreen may also be used to receive input when it is a touch screen. The user interface 121 may also include input elements, such as a keyboard, button, knobs or the like.

The management device 104 may interface with a network 124, such as a LAN or WAN or combination of such networks. The management device 104 may communicate over network 124 with one or more servers or cloud services 128. The role that the one or more servers or cloud services 128 may play in the exemplary embodiments will be described in more detail below.

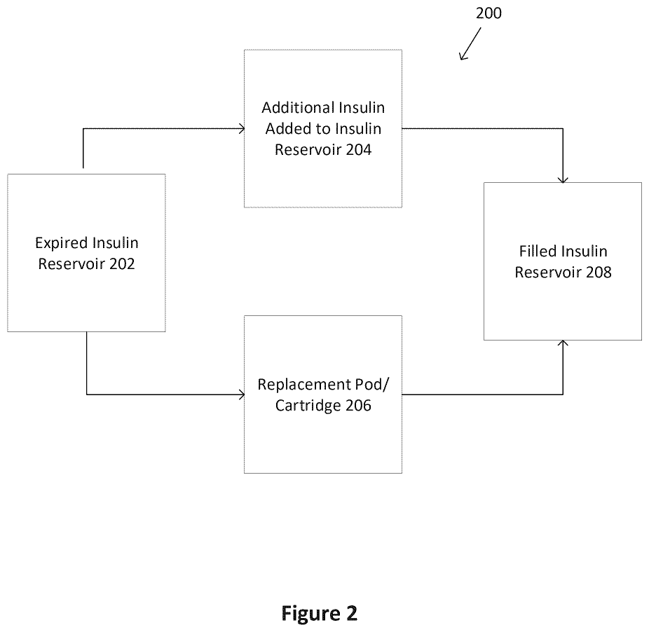

FIG. 2 depicts a block diagram 200 of a situation encountered with insulin delivery systems. When the amount of insulin in an insulin reservoir gets too low, the insulin reservoir is deemed to have expired and needs filling or replacement. Thus, in FIG. 2, expired reservoir 202 represents such an insulin reservoir that has expired. The expired reservoir 202 may be refillable, such that the insulin reservoir structure remains intact but additional insulin in added to the insulin reservoir 204. Another alternative is the that insulin is stored is a discardable pod or cartridge that is disposed of at the time of expiration. A replacement pod or cartridge 206 preloaded with insulin is added to the insulin delivery device 102. The result of both of these approaches is a filled insulin reservoir 208.

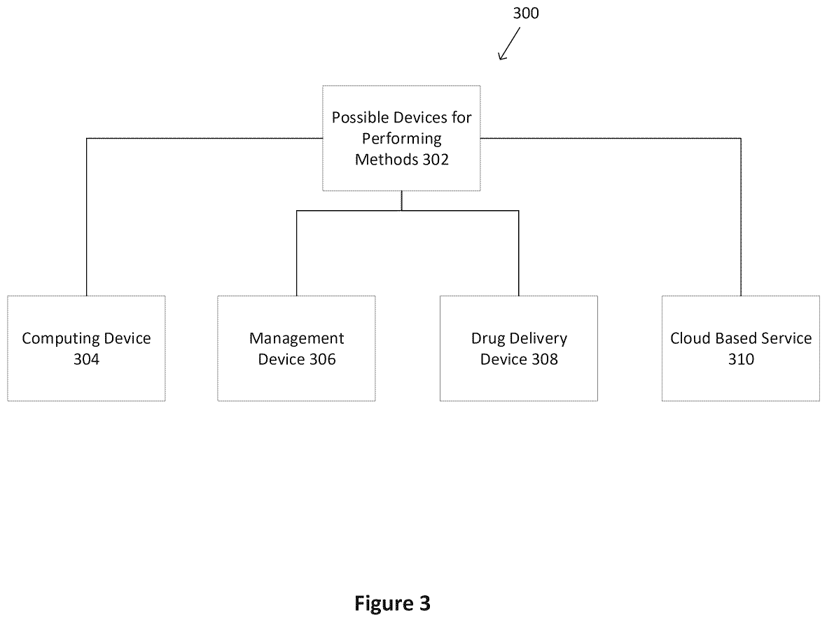

The methods described herein for the exemplary embodiments relating to determining an accurate estimate of a user's daily insulin needs, determining how much insulin to add to an insulin reservoir to fill the reservoir for a user and determining how long before an insulin reservoir will expire may be performed by a number of different types of devices as shown in the diagram 300 of FIG. 3. FIG. 3 depicts possible devices 302 for performing such methods. These methods may be performed by a computing device 304. The computing device may be, for example, computing device 126 that is interfaced with network 122 or a server 128 interfaced with network 124. The methods may also be performed by a management device 306, such as management device 104. Further, the methods may be performed by a drug delivery device 308, such as insulin delivery device 102. Still further, the methods may be performed by a cloud based service 310 like cloud based services 128 accessible on network 124.

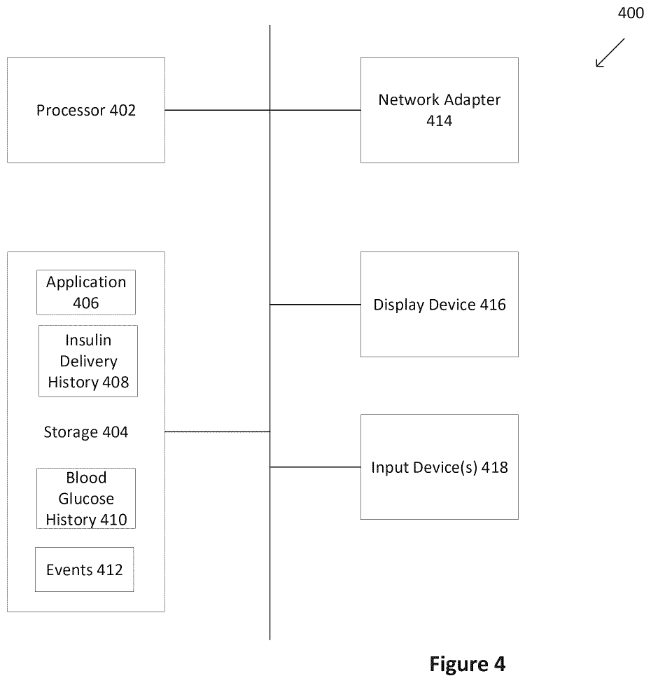

FIG. 4 depicts a block diagram of a device 400 suitable for performing the methods that will be described in more detail below. The device 400 includes a processor 402 for executing programming instructions. The processor 402 has access to a storage 404. The storage 404 may store an application 406 for performing the methods. This application 406 may be executed by the processor 402. The storage 404 may store an insulin delivery history 408 for the user. The insulin delivery history 408 may contain data regarding the amount of insulin delivered as well as the date and time of the deliveries. The insulin delivery history 408 may also identify if each delivery is a basal delivery or a bolus delivery. The storage 404 may store the blood glucose history 410. The blood glucose history 410 may include blood glucose concentration level readings as well as the date and time of such readings. These values may be obtained by the glucose monitor 106. The storage 404 additionally may store information regarding events 412, like meal events and exercise events.

The device 400 may include a network adapter 414 for interfacing with networks, like networks 122 and 124. The device 400 may have a display device 416 for displaying video information. The display device 416 may be, for instance, a liquid crystal display (LCD) device, a light emitting diode (LED) device, etc. The device 400 may include one or more input devices 418 for enabling input to be received. Examples of input devices include keyboards, mice, pointing devices, touchscreen displays, button, knobs or the like.

As was mentioned above, the exemplary embodiments may better estimate a user's daily insulin needs. In some conventional insulin delivery systems, the TDI for a user may be estimated based on factors such as body weight, age, lifestyle or other demographics. The TDI may then, in turn, be used to estimate clinical parameters like basal insulin needs, insulin to carbohydrate (I:C) ratios and correction factors. Unfortunately, calculating TDI in such a fashion is error prone and may not accurately reflect a user's daily insulin needs. The exemplary embodiments may seek to overcome this shortcoming of conventional systems by more accurately estimating daily insulin needs of a user. The exemplary embodiments rely upon the actual insulin delivery history of the user to determine the estimate of daily insulin needs.

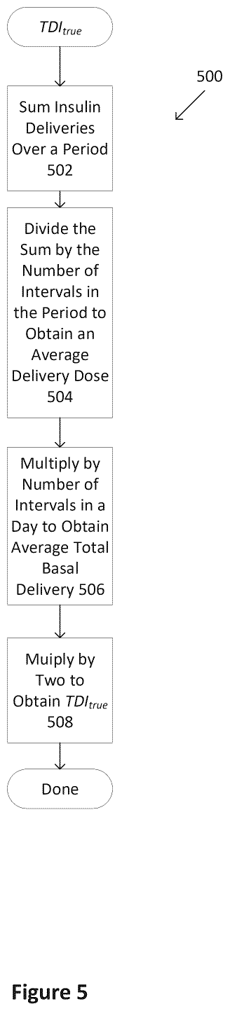

A first aspect of determining the daily insulin needs is to determine a more accurate TDI value than a conventionally calculated TDI. The exemplary embodiments look to the actual automated insulin delivery history to obtain this value. FIG. 5 depicts a flowchart of steps (500) that may be performed to obtain the improved TDI value designated as TDIₜᵣᵤₑ. As a first step, the dosages of automated insulin deliveries to the user from the insulin delivery device 102 over a period of time (L times the length of an interval) are summed (502). This value is divided by L (i.e., the number of intervals in the period of time) to obtain an average insulin delivery for the period (504). The resulting average automated delivery dosage for an interval (such as 5 minutes) is multiplied by the number of intervals in a day to obtain the average total basal insulin delivery per day (506). The number of intervals depends on how often automated insulin delivery is delivered. If the interval is 5 minutes, there are 12 intervals per hour and 24 hours in a day. In such a case, the number of intervals in a day is 12×24 or 288. Hence, for that case, the average automated delivery dosage per interval is multiplied by 288 to obtain the total basal insulin delivery per day. The rule of thumb that TDI is 2 times basal may be applied to obtain TDIₜᵣᵤₑ by multiplying the value calculated in (506) by two (508).

The resulting equation may be expressed as:

$$T D {I}_{true} = 2 \frac{{\sum }_{i = 1}^{L} {I}_{a u t o} ( i )}{L / 288}$$

where Iₐᵤₜₒ(i) is the automated insulin delivery dosage at interval I.

With the estimate TDIₜᵣᵤₑ, the exemplary embodiments may estimate accurately an amount of insulin needed to fill a reservoir to match a user's estimated insulin needs over a period, such as three days. This period may be dictated by a length of time that is safe for the reservoir to hold insulin or a predetermined time assumed by insulin delivery device design. The amount of insulin to be filled into the insulin reservoir may be expressed as:

$${I}_{reservoir} = Number of days ( daily basal needs + daily bolus needs ) .$$

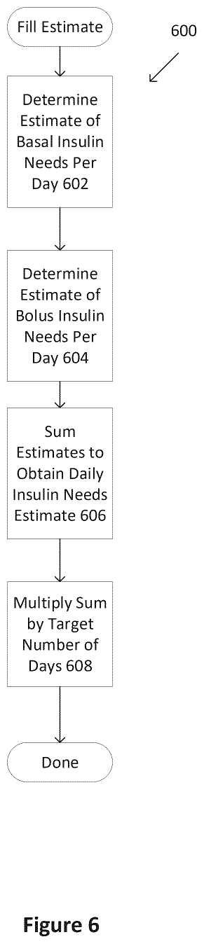

FIG. 6 depicts a flowchart 600 depicting steps that may be performed to determine Iᵣₑₛₑᵣᵥₒᵢᵣ. First, the daily basal needs of the user are determined (602) by looking to the actual average automated insulin deliveries over a period of time (L intervals) and multiplying the average by the number of intervals in a day. If the interval is 5 minutes, then the average is multiplied by 288. Thus, the daily basal needs may be expressed as:

$$\frac{{\sum }_{i = 1}^{L} {I}_{auto} ( i )}{L / 288}$$

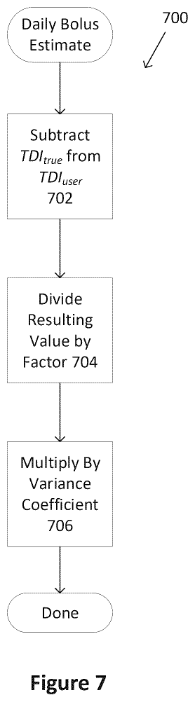

The daily bolus needs may be determined (604). The daily bolus needs may be determined by performing the steps in the flowchart 700 of FIG. 7. The process of determining the daily bolus needs begins by calculating the difference between TDIᵤₛₑᵣ and TDIₜᵣᵤₑ (702). TDIᵤₛₑᵣ is the average daily sum of all insulin deliveries (basal and bolus) for the user. The difference is divided by a factor, such as 2, (704) because it is assumed that half of TDI is attributable to basal. This factor may, however, be varied based on lifestyle or insulin sensitivity of the user. The resulting value may be multiplied by a variance coefficient (706) to account for variability of boluses, such as a value of 1.2, to obtain the average daily bolus needs. The average daily bolus needs may be expressed as:

$$1.2 \frac{{TDI}_{user} - {TDI}_{true}}{2}$$

As expressed above, the daily basal needs and the daily bolus needs are summed (606) to obtain the average daily insulin needs. This sum may then be multiplied by the number of days the insulin is intended to last (608), such as was discussed above. If the insulin is intended to last for three days, the sum is multiplied by three.

Hence, Iᵣₑₛₑᵣᵥₒᵢᵣ may be expressed as:

$${I}_{reservoir} = 3 ( \frac{{\sum }_{i = 1}^{L} {I}_{auto} ( i )}{L / 288} + 1.2 \frac{{TDI}_{user} - {TDI}_{true}}{2} )$$

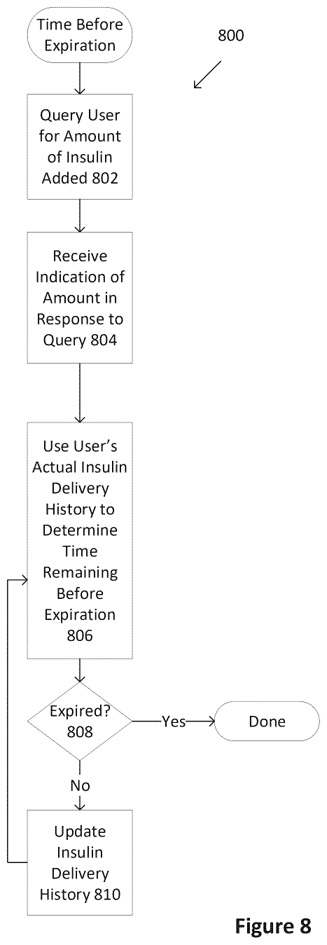

The exemplary embodiments also may estimate how long an amount of insulin in an insulin reservoir will last before being totally consumed by the user. The estimate may be based on the actual insulin use history of the user. FIG. 8 depicts a flowchart 800 of steps that may be performed to determine the estimate of how long before the insulin reservoir expires (i.e., will be consumed). At the time of filling the insulin reservoir, the user is queried (such as via a user interface) to provide an amount of insulin in the filled reservoir (802). The amount of insulin is received in response to the query (804). The user's actual insulin delivery history to determine the estimate of time remaining (806). This may be expressed as:

$$Days of insulin remaining = Amount filled / ( \frac{{\sum }_{i = 1}^{L} {I}_{auto} ( i )}{L / 288} + 1.2 \frac{{TDI}_{user} - {TDI}_{true}}{2} )$$

In some exemplary embodiments, a check may be made whether the insulin reservoir has expired or not (808). This may entail a simple equation for the amount estimated to be remaining using a variation of the formula

$$i . Amount remaining = Amount filled - Number of days since filling ( \frac{{\sum }_{i = 1}^{L} {I}_{auto} ( i )}{L / 288} + 1.2 \frac{{TDI}_{user} - {TDI}_{true}}{2} )$$

Expiration may be reached when the estimated amount remining is below a threshold. Alternatively, a time test could be used. For example, one the determined days of insulin remaining is reached, the reservoir is deemed expired. The value for days remaining may be expressed as a digital value (e.g., 2.7 days) or may be expressed instead in hours or even minutes. If the insulin reservoir has not expired (see 808), the insulin delivery history may be updated each time a delivery is made so that the estimate of insulin remaining is accurate on an ongoing basis (810), and the steps may be repeated after the update beginning at (806) until expiration is reached.

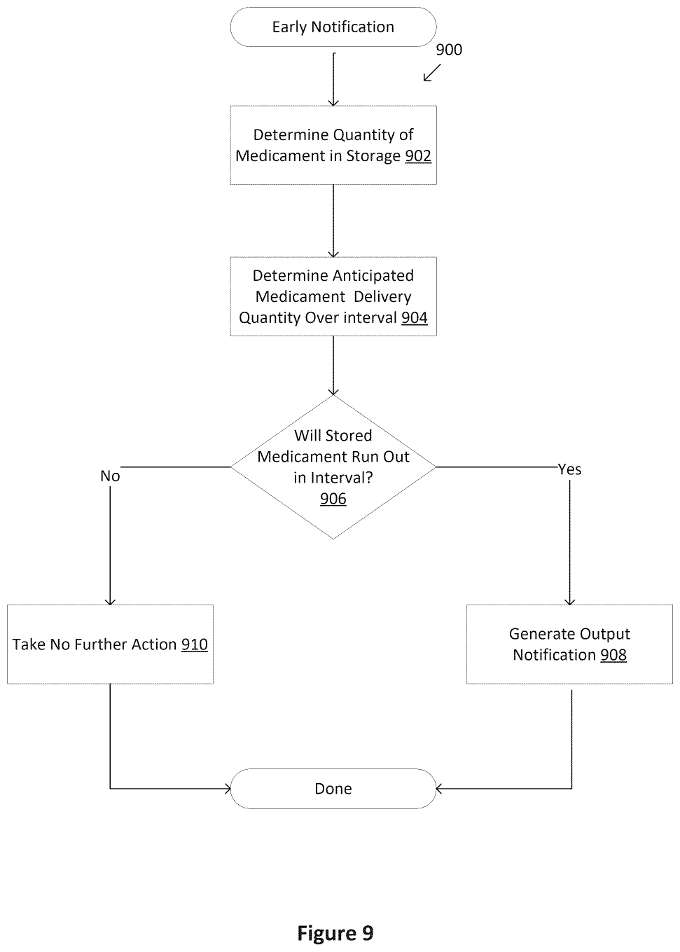

The exemplary embodiments may provide the user with timely alerts that the medicament in a medicament storage, such as insulin reservoir 112, is due to run out within an interval, such as a period of a hour or more. These alerts may be provided with sufficient lead time to enable a user to prepare for the medicament being fully consumed. For instance, the alerts may provide the user with sufficient lead time to refill the medicament in the reservoir or to replace the cartridge, vial, or other storage for the medicament. FIG. 9 depicts a flowchart 900 of illustrative steps that may be performed in exemplary embodiments to provide such early notifications. At 902, the control application 116 or 123 may determine the quantity of medicament in the medicament storage. As will be explained in more detail below, the liquid volume sensor 125 may provide information regarding the quantity of medicament in the medicament storage. At 902, the control application 116 or 123 may determine the anticipated quantity of medicament to be delivered to the user by the medicament delivery system over the interval. The interval may be, for example, a six hour interval, or other durations. At 906, a check is made of whether the anticipated insulin delivery quantity from the medicament delivery system is greater than or equal to the quantity of medicament in the storage. If so, a notification, such as an alert or alarm, may be output at 908. The notification may be textual output that is output via a display or printer. The notification may be an audio output that is output on a speaker. The notification may be include both audio and text. The notification may include graphical output as well or instead of the other varieties of output. The notification output may include tactile output, such as a vibration. The notification may inform the user that the medicament is likely to run out in the interval and/or that there is a need to add medicament to the storage or replace a cartridge or other storage containing the medicament. If the medicament is not anticipated to run out, at 910, no further action is taken.

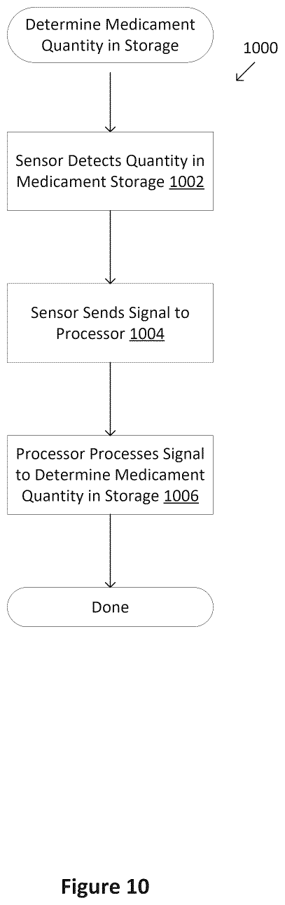

FIG. 10 depicts a flowchart 1000 of illustrative steps that may be performed in exemplary embodiments to determine the medicament quantity in the medicament storage (see 902). At 1002, a liquid volume sensor 125 may be provided within or in communication with the medicament storage, such as insulin reservoir 112, and the liquid volume sensor 125 may detect a volume of medicament in the medicament storage. The liquid volume sensor 125 may be, for example, a capacitive sensor, a float sensor, an ultrasonic sensor, a differential pressure sensor, etc. At 1004, the liquid volume sensor 125 may generate and send a signal to the one or more processors 110 or 119. The signal may encode information indicative of the quantity of medicament sensed in the medicament storage. The volume sensor may generate a signal indicative of the liquid volume in the medicament storage. This liquid volume amount may equate with the quantity of medicament in the medicament storage. At 1006, the one or more processors 110 (designated as controller 110) or 119 may receive the signal and process the signal to determine the quantity of medicament in the medicament storage. In some embodiments, the controller 110 of the insulin delivery device may process the signal to determine the quantity of medicament in the medicament storage, whereas in other embodiments the processor 119 of the management device 104 may process the signal to determine the quantity of medicament in the medicament storage.

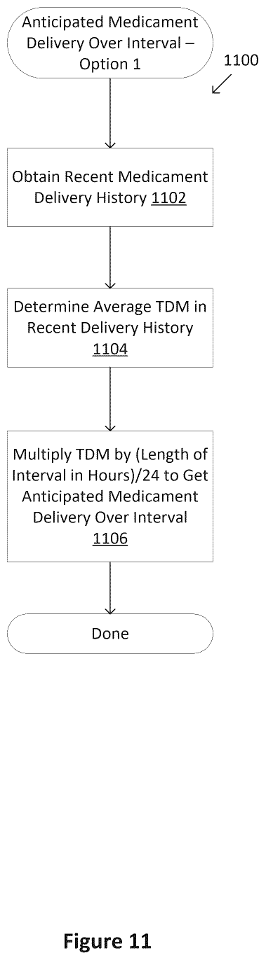

As discussed above, at step 904, the anticipated quantity of medicament delivery over the interval may be determined. FIG. 11 depicts a flowchart 1100 of illustrative steps that may be performed in exemplary embodiments to determine the anticipated quantity of medicament delivery over the interval in accordance with a first option. At 1102, the recent medicament delivery history may be obtained. For example, the recent medicament delivery history may be stored in a database, such as histories 113 in storage 114 or histories 120 in storage 118, that is accessed by the one or more processors 110 or 119. At 1104, the average total daily medicament (TDM) in the recent delivery history is determined. Where the medicament is insulin, TDM equates with the total daily insulin (TDI). The TDM is the aggregate sum of all quantities of medicament delivered to the user in a day. The recent medicament history in histories 113 or 120 may hold the delivery history quantities for a period that extends at least one or more days. The TDM value may be the average TDM for the days in the recent medicament delivery history. The length of the recent medicament delivery history may vary and is a configurable value. Where the medicament is insulin, the recent medicament delivery history may contain data for durations, for example, in the range between 1 day and 7 days, although other durations may be used.

At 1106, the anticipated medicament delivery over the interval may be expressed as

$$N \cdot TDM / 24$$

where N is the number of hours in the interval. This calculation determines the hourly medicament delivery rate from the TDM and then multiplies that rate by the number of hours in the interval.

When such an approach to determining the anticipated quantity of medicament delivery over the interval is used, the triggering of an alert as captured by the binary variable AL_{N}(i) at cycle i may be expressed as

$${Al}_{N} ( i ) = \{ \begin{matrix}1 & R ( i ) \leq N \cdot \frac{TDM}{24} \\ 0 & otherwise\end{matrix}$$

where R(i) is the quantity (i.e., liquid volume) of the medicament remaining in the medicament storage, 1 indicates that an alert is to be generated and 0 indicates that an alert is to not be generated.

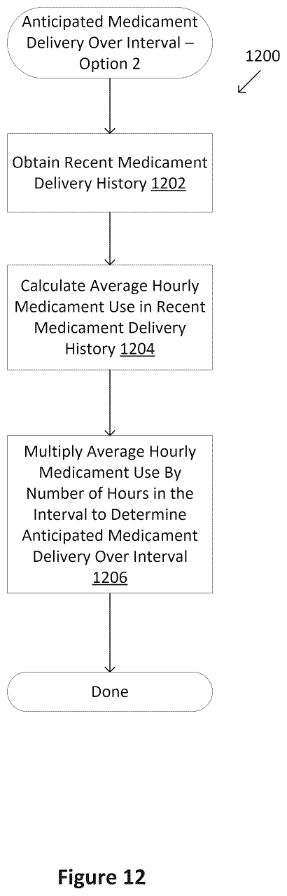

The anticipated medicament delivery over the interval N may instead be determined in exemplary embodiments in accordance with a second option as depicted in the flowchart 1200 of FIG. 12. At 1202, recent medicament delivery history is obtained, such as from histories 113 in storage 114 or from histories 120 in storage 118. At 1204, the controller 110 or processor 119, may calculate the average hourly medicament use in the recent medicament delivery history. This may be calculated as:

$$\frac{{\sum }_{j = 1}^{M \cdot 12} I ( i - j )}{M}$$

where M is a period of hours in the obtained recent medicament delivery history, j is a cycle index, i is the current cycle index, and I(i−j) is the quantity of medicament delivered in the cycle i−j. M is multiplied by 12 in the above equation because cycles are presumed to be 5 minutes in length so there are twelve cycle per hour. It should be appreciated that cycle lengths may be other durations, such as durations in the range of 1 to 15 minutes. At 1206, the average hourly medicament use is multiplied by the number of hours in the interval (i.e., N). When the second option is used in determining the anticipated insulin delivery over the period, the value of the binary variable Al_{N}(i) may be expressed as:

$${Al}_{N} ( i ) = \{ \begin{matrix}1 & R ( i ) \leq N \cdot \frac{{\sum }_{j = 1}^{M \cdot 12} I ( i - j )}{M} \\ 0 & otherwise\end{matrix}$$

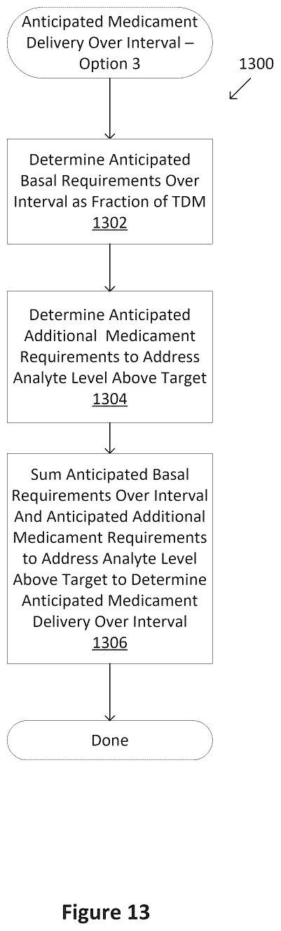

The anticipated medicament delivery medicament delivery over the interval may in some exemplary embodiments be determined by a third approach. FIG. 13 depicts a flowchart 1300 of illustrative steps that may be performed in exemplary embodiments in accordance with the third approach. At 1302, the anticipated basal requirements of the user over the interval may be determined as a fraction of TDM. At 1304, the additional medicament requirements to address the analyte level of the user being above a target are determined. Where the medicament is insulin and the analyte level is the glucose level of the user, the additional requirements correspond to the amount of insulin needed to bring the glucose level of the user to the target glucose level. With other medicaments, the additional requirements may constitute a quantity of medicament to bring a current analyte level of the user to a target level. At 1306, The anticipated medicament delivery over the interval may be calculated as the sum of the anticipated basal medicament delivery requirements over the interval and the anticipated additional medicament requirements to address the analyte level being above a target level.

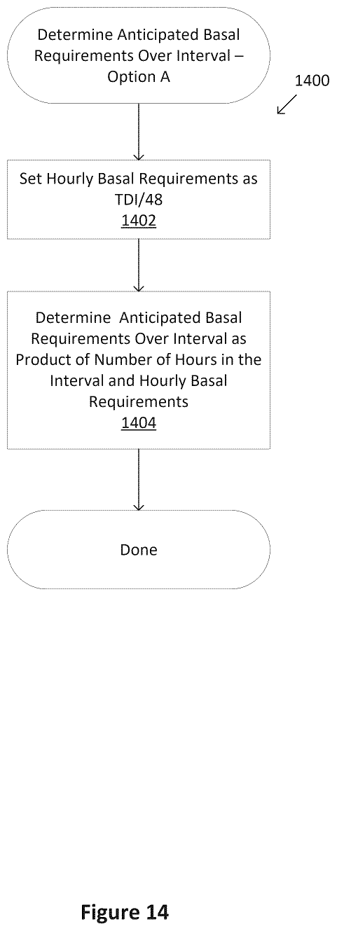

In accordance with a first option, designated as option A herein, which is depicted in the flowchart 1400 of FIG. 14, the anticipated basal requirements per hour for insulin as an example medicament may be determined as

$$\frac{TDI}{48}$$

(i.e., ½ of TDI/24, the hourly insulin delivery rate derived from TDI) at 1402. At 1404, The anticipated basal requirements over the interval may be determined as the product of the number of hours in the interval (i.e., N) and the hourly basal requirements. In other words, the product may be calculated as

$$N \cdot \frac{TDI}{48} .$$

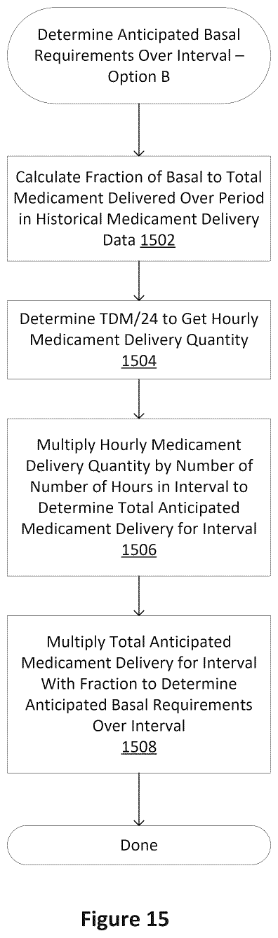

The anticipated basal requirements over the interval may also be calculated per a second option, designated as option B herein. FIG. 15 depicts a flowchart 1500 of illustrative steps that may be performed in exemplary embodiments per option B. At 1502, the histories 113 or 120 may be accessed, and the delivery data stored therein may be processed to determine the ratio of basal medicament delivered to the user over a period relative to the total medicament delivered to the user over the interval (the ratio is designated as P). This ratio P may be calculated as:

$$P ( i ) = \frac{{\sum }_{j = 1}^{M \cdot 12} {I}_{b} ( i - j )}{{\sum }_{j = 1}^{M \cdot 12} I ( i - j )}$$

where j is a cycle index, i is an index of the current cycle, M is the number of cycles per hour, I(i−j) is the quantity of all insulin delivered over cycle i−j, and I_{b}(i−j) is the quantity of basal insulin delivered over cycle i−j.

At 1504, the hourly medicament delivery quantity may be determined by dividing the average TDM for the period by 24. At 1506, the hourly medicament delivery quantity may be multiplied by the number of hours N in the interval

$$( i . e . , N \cdot \frac{TDI}{24} )$$

to determine the total anticipated medicament delivery for the interval. At 1508, the total anticipated medicament delivery for the interval may be multiplied by the ratio P to determine the anticipated basal requirements over the interval. Thus, the anticipated basal requirements over the interval may be expressed as

$$N \cdot P ( i ) \cdot \frac{TDI}{24} .$$

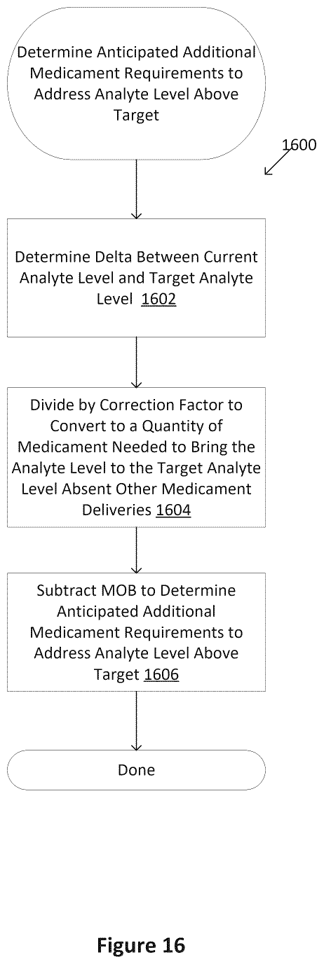

FIG. 16 depicts a flowchart 1600 of illustrative steps that may be performed in determining the anticipated additional medicament requirements to address the analyte level of the user being above a target (see 1404 of FIG. 14). At 1602, a delta between the current analyte level for the user and a target analyte level for the user may be determined. For example, a difference between a current glucose level of a user and a target glucose level of a user may be determined. At 1604, the determined delta may be converted into a quantity of medicament needed to bring the analyte level of the user to the target level absent other medicament deliveries. Where the medicament is insulin and the analyte level is glucose level, this calculation may be expressed as (CGM(i)−SP(i))/CF), where CGM(I) is the glucose level reading for cycle i, SP(i) is the target glucose level for cycle I, and CF is the correction factor. In some embodiments, the correction factor may be 1800/TDI or a similar value. At 1606, the medicament on board (MOB) is subtracted from (CGM(i)−SP(i))/CF to account for the insulin that has already has been delivered that has not yet been fully metabolized and that hence can still reduce glucose levels.

When this third option for calculating the anticipated medicament delivery over the interval is used, the value of the binary variable Al_{N}(i) may be expressed as:

$${Al}_{N} ( i ) = \{ \begin{matrix}1 & R ( i ) \leq N \cdot P ( i ) \cdot \frac{TDI}{24} + \frac{CGM ( i ) - SP ( i )}{1800 / TDI} - IOB ( i ) \\ 0 & otherwise\end{matrix}$$

While the present invention has been described with reference to exemplary embodiments described herein, it should be appreciated that various changes in form and detail may be made without departing from the intended scope of the present invention as defined by the claims appended hereto.

1. A medicament delivery system for delivering a medicament to a user, comprising:
a medicament storage for storing the medicament;
an output element for generating output to the user;
a non-transitory storage medium storing programming instructions;
one or more processors for executing the programming instructions to cause the one or more processors to:
determine an amount of the medicament that is stored in the medicament storage,
determine a quantity of the medicament that will delivered to the user over a period by the medicament delivery system based at least in part on a current analyte level of the user,
determine whether the quantity of medicament that will be delivered to the user over the period by the medicament delivery system exceeds the amount of medicament that is stored in the medicament storage, and
generate output on the output element where it is determined that the quantity of medicament that will be delivered to the user over the period by the medicament delivery system exceeds the amount of medicament that is stored in the medicament storage.

2. The medicament delivery system of claim 1, wherein the determining of the quantity of the medicament that will delivered to the user over a period by the medicament delivery system is based in part on a difference between the current analyte level of the user and a target analyte level of the user.

3. The medicament delivery system of claim 2, wherein the determining of the quantity of the medicament that will delivered to the user over a period by the medicament delivery system is additionally based on a quantity of medicament that has been delivered to the user that still has an effect on the analyte level of the user.

4. The medicament delivery system of claim 3, wherein the analyte level is a glucose level of the user.

5. The medicament delivery system of claim 4, wherein the medicament is insulin and wherein the quantity of medicament that has been delivered to the user that still has an effect on the analyte level of the user is insulin on board (IOB).

6. The medicament delivery system of claim 1, wherein the determining of the quantity of the medicament that will delivered to the user over a period by the medicament delivery system is based in part on a total medicament delivered to the user over a prior period.

7. The medicament delivery system of claim 1, wherein the determining of the quantity of the medicament that will delivered to the user over a period by the medicament delivery system is based in part on a ratio of basal insulin delivered to the user over a prior period to a total of insulin delivered to the user over the prior period.

8. An insulin delivery system for delivering insulin to a user, comprising:
a storage for storing the insulin;
an output element for generating output to the user;
a non-transitory storage medium storing programming instructions;
one or more processors for executing the programming instructions to cause the one or more processors to:
receive from a sensor a signal indicative of an amount of insulin stored in the storage;
determine from the signal how much insulin is stored in the storage;
determine a quantity of the insulin that will delivered to the user over a period by the insulin delivery system based at least in part on a current glucose level of the user, a target glucose level of the user and insulin on board for the user;
generate output on the output element where it is determined that the quantity of insulin that will be delivered to the user over the period by the insulin delivery system exceeds the amount of insulin that is stored in the storage.

9. The insulin delivery system of claim 8, wherein the one or more processors in determining the quantity of insulin that will be delivered over the period determine an amount of insulin that is needed to bring the current glucose level of the user to a target level of the user.

10. The insulin delivery system of claim 9, wherein the one or more processors in determining the quantity of insulin that will be delivered over the period reduce the determined amount of insulin that is needed to bring the current glucose level of the user to a target level of the user by the insulin on board for the user.

11. The insulin delivery system of claim 10, wherein the one or more processors in determining the quantity of insulin that will be delivered over the period add an anticipated amount of basal insulin to be delivered over the period based on a total daily insulin (TDI) value derived from recent insulin delivery data for the user.

12. The insulin delivery system of claim 11, wherein the anticipated amount of basal insulin to be delivered over the period based on the TDI value for the user is set as half of the TDI value for the user.

13. The insulin delivery system of claim 11, wherein the anticipated amount of basal insulin to be delivered over the period based on the TDI value for the user is set as a product of multiplying the TDI value for the user by a proportionality value that is indicative of what portion of the TDI for the user are basal insulin deliveries in the recent insulin delivery data for the user.

14. The insulin delivery system of claim 8, wherein the output is a notification that the insulin in the storage will be fully consumed by a specified date.

15. A medicament delivery system for delivering a medicament to a user, comprising:
a medicament storage for storing the medicament;
an output element for generating output to the user;
a non-transitory storage medium storing programming instructions;
one or more processors for executing the programming instructions to cause the one or more processors to:
determine a quantity of medicament that remains in the medicament storage;
determine a quantity of the medicament will be delivered from the medicament storage over an interval of at least an hour in duration, comprising:
determining a quantity of basal medicament that will be delivered over the interval as basal medicament deliveries,
determining a quantity of medicament for a correction bolus that would bring an analyte level of the user to a target level, and
summing the determined quantity of basal medicament that will be delivered over the interval as basal medicament deliveries with the detrained quantity for the correction bolus to determine a sum used in determining the quantity of the medicament will be delivered from the medicament storage over the interval,
comparing the quantity of the medicament that will be delivered from the medicament storage over the interval to the quantity of the medicament that remains in the medicament storage,
where the quantity of the medicament that will be delivered from the medicament storage over the interval is greater than the quantity of the medicament that remains in the medicament storage, generating a notification to the user, and
where the quantity of the medicament that will be delivered from the medicament storage over the interval is not greater than the quantity of the medicament that remains in the medicament storage, taking no additional action.

16. The medicament delivery system of claim 15, wherein the medicament is insulin, and the analyte level is a glucose level.

17. The medicament delivery system of claim 15, wherein the determining of the quantity of basal medicament that will be delivered over the interval as basal medicament deliveries comprises analyzing recent basal medicament delivery history of the user to determine the quantity of basal medicament that will be delivered over the interval as basal medicament deliveries.

18. The medicament delivery system of claim 17, wherein the determining of the quantity of basal medicament that will be delivered over the interval as basal medicament deliveries comprises calculating an average total daily medicament for the user from the recent basal medicament delivery history of the user and determining a daily quantity of the basal medicament that will be delivered over the interval as fraction of the average total daily medicament for the user.

19. The medicament delivery system of claim 18, wherein the medicament is insulin and the fraction is one of either one half or a fraction derived from the recent basal medicament delivery history.

20. The medicament delivery system of claim 15, wherein determining of the quantity of the medicament that will delivered from the medicament storage over the interval further comprises subtracting from the sum quantities of medicament that have been delivered to the user that may still affect the analyte level of the user.
